(19) United States
(12) Patent Application Publication
(10) Pub. No.: US 2015/0141818 A1
(43) Pub. Date: May 21, 2015

(54) VASCULAR IMAGING METHOD AND DEVICE

(71) Applicant: Shenyang Neusoft Medical Systems Co., Ltd., Shenyang (CN)

(72) Inventors: Jian ZHAO, Shenyang (CN); Bowen ZHANG, Shenyang (CN)

(21) Appl. No.: 14/527,760

(22) Filed: Oct. 29, 2014

(30) Foreign Application Priority Data
Nov. 21, 2013 (CN) 201310594580.7

(51) Int. Cl. A61B 6/00; A61B 6/03; A61B 5/055; A61B 5/00

(52) U.S. Cl. CPC A61B 6/504; A61B 5/489; A61B 5/7425; A61B 6/032; A61B 6/037; A61B 5/055; A61B 6/466; A61B 6/5252; A61B 6/469; A61B 5/748; A61B 6/5241; A61B 6/501; A61B 6/5217; A61B 6/481
USPC 600/431

(57) ABSTRACT

A vascular imaging method and device are provided according to the embodiments of the present disclosure. The method includes: scanning a region where a vessel is located to obtain a noncontrast enhanced scan image and a contrast enhanced image, and performing subtraction on the contrast enhanced image by using a bone region in the noncontrast enhanced scan image to obtain a subtraction image; then detecting a vascular region of the vessel in the contrast enhanced image; finally, combining the subtraction image with the vascular region of the vessel to obtain an angiography image of the vessel. According to the present disclosure, the vascular region in which the vessel passes through the bone is maintained in the subtraction image.

### CROSS REFERENCE TO RELATED APPLICATIONS

This application claims priority to Chinese Patent Application No. 201310594580.7, entitled “VASCULAR IMAGING METHOD AND DEVICE”, filed with the Chinese State Intellectual Property Office on Nov. 21, 2013, which is incorporated herein by reference in the entirety.

### FIELD

The present disclosure relates to the field of medical imaging, and in particular to a vascular imaging method and device.

### BACKGROUND

There has been an increasing demand for vascular imaging technology because vascular disease is one of the diseases seriously affecting human health. Due to the complexity of the human vascular morphology, part of a vessel may be blocked by a bone. Therefore, it may be difficult to visually display the complete morphology and structure of the vessel in three-dimensional volume rendering. For example, the vessel in the head and neck region starts from the heart and goes into the brain through the bone of skull base; the carotid artery passes through the skull; such as the vertebral artery passes through six vertebras and enters into the skull.

Presently, the vascular region is maintained generally by subtracting a bone region from an image using the subtraction angiography. The existing subtraction angiography is implemented by scanning a patient twice and the patient has to be injected with intravascular contrast media for the second scan, which leads to a long time interval between the first scan and the second scan. Because it is difficult for the patient to maintain a same posture for a long time, there may be displacement of the detected region of the patient during the two scans, thus causing a problem that the part of the vessel that passes through the bone may be mistaken for the bone region and subtracted. Therefore the subtraction image generated by using the existing subtraction angiography cannot display the structure of the vascular region accurately and completely.

### SUMMARY

In order to solve the technical problem described above, a vascular imaging method and device are provided in the present disclosure, in which an angiography image is obtained by detecting a vascular region of a vessel in a contrast enhanced image, aligning and combining the vascular region with a subtraction image. With this process, the subtracted image of the part of the vessel passing through the bone for may be maintained, therefore the accuracy and reliability of angiography imaging is enhanced.

A technical scheme provided by the present disclosure is as follows.

There is provided a vascular imaging method, including:

scanning a region where a vessel is located to obtain a noncontrast enhanced scan image and a contrast enhanced image, and performing subtraction on the contrast enhanced image by using a bone region in the noncontrast enhanced scan image to obtain a subtraction image;

detecting a vascular region of the vessel in the contrast enhanced image; and

combining the subtraction image with the vascular region of the vessel to obtain an angiography image of the vessel.

Where the detecting a vascular region of the vessel in the contrast enhanced image includes:

estimating a vascular path of the vessel in the contrast enhanced image based on vascular grayscale distribution;

determining a vascular radius at each point on the vascular path based on grayscale smoothness; and

segmenting the contrast enhanced image according to the vascular radius and the vascular path to obtain the vascular region of the vessel.

Where the estimating a vascular path of the vessel in the contrast enhanced image based on vascular grayscale distribution includes:

matching the contrast enhanced image and a model of the bone region through which the vessel passes to determine a position of a bone in the contrast enhanced image.

estimating a region of interest where the vessel is located based on the position of the bone, matching contrast enhanced images of the region of interest with a template of vascular cross-sectional grayscale distribution layer-by-layer to determine start points and end points of the vessel; and

calculating a grayscale similarity between each point in the contrast enhanced image and the start points and end points of the vessel, and selecting points with a minimum grayscale similarity to constitute a vascular path of the vessel.

Where the determining a vascular radius at each point on the vascular path based on grayscale smoothness includes:

calculating the grayscale smoothnesses within different radiuses with respect to the each point on the vascular path; and

selecting the largest radius which meets a smoothness threshold as the vascular radius at the each point.

Where the estimating a region of interest where the vessel is located based on the position of the bone, matching contrast enhanced images of the region of interest with a template of vascular cross-sectional grayscale distribution layer-by-layer to determine start points and end points of the vessel includes:

establishing the template of vascular cross-sectional grayscale distribution at least one scale in advance;

estimating the region of interest where the vessel is located based on the position of the bone and matching the contrast enhanced image of the region of interest with the vascular cross-sectional grayscale distribution template layer-by-layer to obtain potential positions of the vessel in the region of interest; and

positioning and clustering the potential positions of the vessel in the region of interest by using a clustering algorithm and selecting points in a maximum cluster as the start points and end points of the vessel based on a positional relationship between the vessel and the bone where the vessel is located.

Where in the case that the vessel is in a head and neck region,

the estimating a region of interest where the vessel is located based on the position of the bone, matching the contrast enhanced images of the region of interest with a template of vascular cross-sectional grayscale distribution layer-by-layer to determine a start point and an end point of the vessel includes:

establishing the template of vascular cross-sectional grayscale distribution at least one scale in advance;

estimating the region of interest where the vessel is located based on a position of a skull and matching contrast enhanced images of the region of interest with the template of vascular cross-sectional grayscale distribution layer-by-layer to determine potential end points of the vessel in the region of interest;

estimating a position of the neck based on the position of the skull and detecting contrast enhanced images of the neck region layer-by-layer by using an edge detection algorithm and a circular detection operator to obtain potential start points of the vessel in the region of interest; and

positioning and clustering the potential start points and the potential end points by using clustering algorithm and selecting points in a maximum cluster as the start points and end points of the vessel.

Where the method further includes:

displaying the angiography image of the vessel by using three-dimensional volume rendering.

There is further provided a vascular imaging device, including:

a scanning unit, configured to detect a region where a vessel is located to obtain a noncontrast enhanced scan image and a contrast enhanced image and to perform subtraction on the contrast enhanced image by using a bone region in the noncontrast enhanced scan image to obtain a subtraction image;

a detecting unit, configured to detect a vascular region of the vessel in the contrast enhanced image; and

a combining unit, configured to combine the subtraction image with the vascular region of the vessel to obtain an angiography image of the vessel.

Where the detecting unit includes:

an estimating sub-unit, configured to estimate a vascular path of the vessel in the contrast enhanced image based on vascular grayscale distribution;

a determining sub-unit, configured to determine a vascular radius at each point on the vascular path based on grayscale smoothness; and

a segmenting sub-unit, configured to segment the contrast enhanced image based on the vascular radius and the vascular path to obtain the vascular region of the vessel.

Where the estimating sub-unit includes:

a first matching module, configured to match the contrast enhanced image and a model of the bone region through which the vessel passes to determine the position of the bone in the contrast enhanced image;

a second matching module, configured to estimate a region of interest where the vessel is located based on a position of the bone, and to match contrast enhanced images of the region of interest with a template of vascular cross-sectional grayscale distribution layer-by-layer to determine start points and an end points of the vessel; and

a first selected module, configured to calculate a grayscale similarity between each point in the contrast enhanced image and the start points and end points of the vessel, and to select points with a minimum grayscale similarity to constitute a vascular path of the vessel.

Where the determining sub-unit includes:

a first calculating module, configured to calculate grayscale smoothnesses within different radiuses with respect to the each point on the vascular path; and

a second selecting module, configured to select the largest radius which meets a smoothness threshold as the vascular radius at the each point.

Where the second matching module includes:

an establishing sub-module, configured to establish the template of vascular cross-sectional grayscale distribution at least one scale in advance;

a matching sub-module, configured to estimate the region of interest where the vessel is located based on the position of the bone and to match contrast enhanced images of the region of interest with the template of vascular cross-sectional grayscale distribution layer-by-layer to obtain potential positions of the vessel in the region of interest; and

a selecting sub-module, configured to position and cluster the potential positions of the vessel in the region of interest by using a clustering algorithm and to select points in a maximum cluster as the start points or end points of the vessel based on a positional relationship between the vessel and the bone where the vessel is located.

Where in the case that the vessel is in a head and neck region,

the second matching module includes:

an establishing sub-module, configured to establish the template of vascular cross-sectional grayscale distribution at least one scale in advance;

a matching sub-module, configured to estimate the region of interest where the vessel is located based on a position of a skull and to match the contrast enhanced images of the region of interest with the template of vascular cross-sectional grayscale distribution layer-by-layer to determine potential end points of the vessel in the region of interest;

a detecting sub-module, configured to estimate a position of the neck based on the position of the skull and detect contrast enhanced images of the neck region layer-by-layer by using an edge detection algorithm and a circular detection operator to obtain potential start points of the vessel in the region of interest; and

a selecting sub-module, configured to position and cluster the potential start points and the potential end points by using a clustering algorithm and to select points in a maximum cluster as the start points and end points of the vessel.

Where the device further includes a rendering unit configured to display the angiography image of the vessel by using three-dimensional volume rendering.

For ensuring to display the vascular region of the vessel passing through the bone accurately during vascular imaging, in the vascular imaging method and device according to the present disclosure, firstly the region where the vessel is located is scanned to obtain a noncontrast enhanced scan image and a contrast enhanced image, and subtraction is performed on the contrast enhanced image by using a bone region in the noncontrast enhanced scan image to obtain a subtraction image; then the vascular region of the vessel in the contrast enhanced image is detected. Thereby the vascular region of the vessel in the contrast enhanced image is determined and the vascular region is a mistakenly subtracted vascular region of the part of the vessel that passes through or close to the bone; finally, the subtraction image is combined with the vascular region of the vessel to obtain the angiography image of the vessel. Thus the advantageous effect of the present disclosure is that: the part of the vessel that passes through or close to the bone is maintained by detecting the vascular region of the vessel in the contrast enhanced image and then combining the image of the vascular region of the vessel by using the subtraction image, thereby the structure of the vascular region of the vessel is displayed completely.

### BRIEF DESCRIPTION OF THE DRAWINGS

In order that technical scheme according to the embodiment of the present disclosure or according to the prior art may be illustrated more clearly, the drawings which are needed to be used in the description of the embodiments will be introduced briefly in the following. Apparently, the drawings in the following description are only a few of embodiments of the present disclosure. For the skilled in the art, other drawings may be derived according to those drawings without inventive effort.

FIG. 1 is a flow chart of a vascular imaging method according to a first method embodiment of the present disclosure;

FIG. 2 is a flow chart of a vascular imaging method according to a second method embodiment of the present disclosure;

FIG. 3 is a schematic diagram of a vascular imaging device according to a first device embodiment of the present disclosure; and

FIG. 4 is a schematic diagram of a vascular imaging device according to a second device embodiment of the present disclosure.

### DETAILED DESCRIPTION OF THE EMBODIMENTS

To make the above object, features and advantages of the disclosure more obvious and easy to be understood, in the following, particular embodiments of the disclosure will be explained in detail in conjunction with the drawings.

It should be noted that the method according to the present disclosure may be applied to systems for scanning and imaging the vessel, such as CT (Computed Tomography), PET-CT (Positron Emission Tomography), MRI (Magnetic Resonance Imaging).

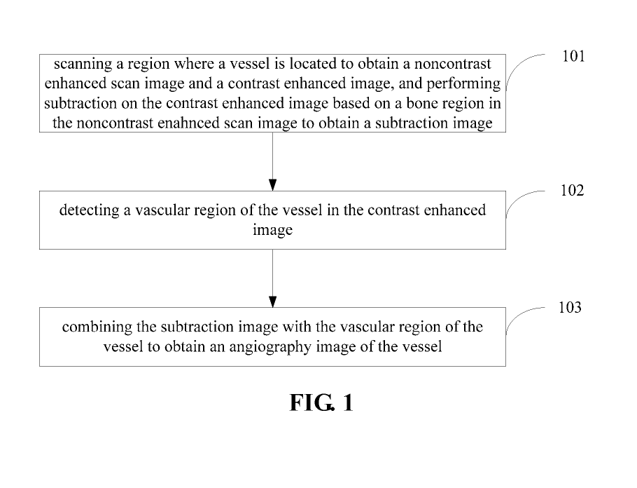

Referring to FIG. 1, a flow chart of a vascular imaging method according to a first method embodiment of the present disclosure is shown. The method includes Step 101 to Step 103.

Step 101 includes scanning a region where a vessel is located to obtain a noncontrast enhanced scan image and a contrast enhanced image, and performing subtraction on the contrast enhanced image based on a bone region in the noncontrast enhanced scan image to obtain a subtraction image.

During an actual scanning process, a corresponding scan region is determined based on the vessel. For example, in the case that the cardiovascular of a patient is to be detected, the thoracic cavity of the patient is scanned; and in the case that the vessel in the head and neck region is to be detected, the head and neck region of the patient is scanned. After the region where the vessel is located is determined, firstly, a transverse image of the region is scanned to obtain a noncontrast enhanced scan image, and a bone region in the noncontrast enhanced scan image may be determined by applying threshold segmentation to the noncontrast enhanced scan image; then, the patient is injected with intravascular contrast medium and the region where the vessel is located is scanned again to obtain a contrast enhanced image. Thereafter the contrast enhanced image and the noncontrast enhanced scan image are aligned by using an image registration method and a subtraction image is obtained by subtracting the bone region from the contrast enhanced image.

Step 102 includes detecting a vascular region of the vessel in the contrast enhanced image.

An implementation is provided for above Step 102 according to the present disclosure, which may include Step 1021 to Step 1023.

Step 1021 includes estimating a vascular path of the vessel in the contrast enhanced image based on vascular grayscale distribution.

Step 1022 includes determining a vascular radius at each point on the vascular path based on grayscale smoothness.

Step 1023 includes segmenting the contrast enhanced image based on the vascular radius and the vascular path to obtain the vascular region of the vessel.

An implementation for determining the vascular path is provided for above Step 1021 according to the present disclosure, which may include Step 1021A to Step 1021C.

Step 1021A includes matching the contrast enhanced image and a model of the bone region through which the vessel passes to determine a position of the bone in the contrast enhanced image.

The model of the bone region in this step may be an active shape model (ASM) or an active appearance model (AAM). The matching operation may employ an optimal matching method, such as a conjugate gradient method, a Powell optimization method.

Step 1021B includes estimating a region of interest where the vessel is located based on the position of the bone, matching contrast enhanced images of the region of interest with a template of vascular cross-sectional grayscale distribution layer-by-layer to determine start points and end points of the vessel.

Step 1021C includes calculating a grayscale similarity between the each point in the contrast enhanced image and the start points and end points of the vessel, and selecting points with a minimum difference of grayscale similarity to constitute the vascular path of the vessel.

An implementation for determining the vascular radius is provided for above Step 1022 according to the present disclosure, which may include Step 1022A to Step 1022B.

Step 1022A includes calculating grayscale smoothness within different radiuses with respect to each point on the vascular path.

Step 1022B includes selecting the largest radius which meets a smoothness threshold as the vascular radius at each point.

Step 103 includes combining the subtraction image with the vascular region of the vessel to obtain an angiography image of the vessel.

The combining in this step may be implemented by filling the image of the vascular region of the vessel into the subtraction image according to the coordinate.

The vascular imaging process according to the present disclosure may be implemented by scanning the region where the vessel is located to obtain a noncontrast enhanced scan image and a contrast enhanced image and performing a subtraction process to the contrast enhanced image to obtain a subtraction image; then detecting the vascular region of the vessel in the contrast enhanced image; finally combining the subtraction image with the vascular region of the vessel to obtain an angiography image of the vessel. According to the present application, the vascular region of the vessel is subtracted by mistake is combined into the subtraction image, thereby the region where the vessel passes through the bone is maintained on the subtraction image and the integrity of the vascular region of the vessel is ensured.

An implementation is further provided for the above Step 1021B according to the embodiments of the present disclosure, which may include:

establishing the template of vascular cross-sectional grayscale distribution at least one scale in advance;

estimating a region of interest of the vessel based on the position of the bone and matching the contrast enhanced images of the region of interest with the template of vascular cross-sectional grayscale distribution layer-by-layer to obtain potential positions of the vessel in the region of interest; and

positioning and clustering the potential positions of the vessel in the region of interest by using a clustering algorithm and selecting positioning points in the maximum cluster as the start points and end points of the vessel based on the positional relationship between the vessel and the bone where the vessel is located.

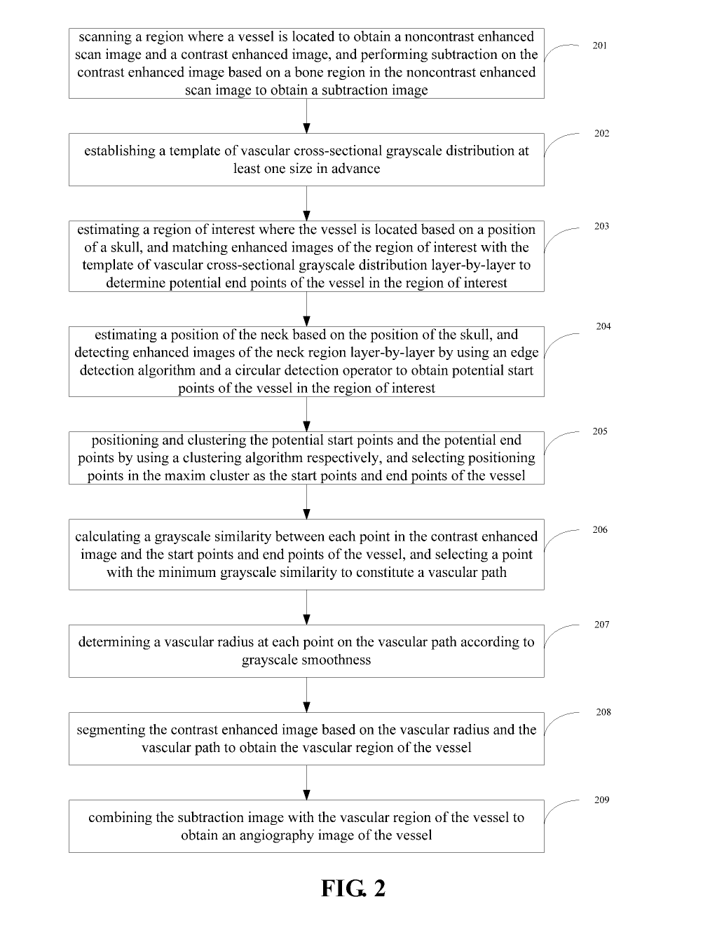

The bones and vessels in different parts of the human body differ in complexity and structural characteristics. For example, the vessels and bones of the head and neck, chest, legs and other parts of the body have completely different structures. The head and neck have the most complicated structure, and the vessel starts from the heart and goes into the brain through the bottom of the skull. There are four main vessels in the head and neck region: the left carotid artery, the right carotid artery, the left vertebral artery and the right vertebral artery. For detecting the four vessels rapidly accurately, the most important step is to determine the start points and the end points of the vessels rapidly and accurately. Therefore, according to a second method embodiment of the present disclosure, it is provided a vascular image method to ensure the accuracy for imaging the vessel in the head and neck region, which is shown in FIG. 2.

A flow chart of a vascular imaging method according to the second method embodiment of the present disclosure includes Step 201 to Step 209.

Step 201 includes scanning a region where a vessel is located to obtain a noncontrast enhanced scan image and a contrast enhanced image, and performing subtraction on the contrast enhanced image based on a bone region in the noncontrast enhanced scan image to obtain a subtraction image.

Step 202 includes establishing a template of vascular cross-sectional grayscale distribution at least one scale in advance.

The vascular radius varies with the different patients. A plurality of templates of vascular cross-sectional grayscale distribution with different scales are obtained, to ensure that the subsequent matching process may guarantee a high accuracy in obtaining a potential central position to meet the needs for scanning different patients. Of course, it is also possible to adopt one template of vascular cross-sectional grayscale distribution at a conventional scale.

Step 203 includes estimating a region of interest where the vessel is located based on a position of a skull, and matching contrast enhanced images of the region of interest with the template of vascular cross-sectional grayscale distribution layer-by-layer to determine potential end points of the vessel in the region of interest.

For four vessels in the skull, the potential end points of the four vessels are estimated based on the position of the skull determined in Step 202. Since a vessel starts from the heart and then enters the brain, the end point of the vessel refers to the position at which the vessel ends, and the start point of the vessel refers to the position on the neck at which the vessel enters the brain.

The position of the artery in the skull may be determined based on anatomic structure experience, and the potential position of the vessel may be determined based on the position of the artery in the skull. For example, the left carotid artery is on the left side of the skull center; the left vertebral artery is on the right side of the skull center; the two vertebral arteries pass through the foramen magnum which is located at the bottom of the skull, the left vertebral artery is on the right side of the left carotid artery and the right vertebral artery is on the right side of the left vertebral artery.

In practice, the intracranial Willis's circle region-of-interest and the basilar artery region-of-interest are often used. The Willis's circle region-of-interest includes: the left arteriae cerebri anterior, the right arteriae cerebri anterior, the left anterior communicating artery region-of-interest and the right anterior communicating artery region-of-interest. The left carotid artery extends on the left side of the left arteriae cerebri anterior and left anterior communicating artery region-of-interest, and the right carotid artery extends on the right side of the right arteriae cerebri anterior and right anterior communicating artery region-of-interest. The arteriae basilaris region-of-interest is formed by the left vertebral artery and the right vertebral artery merging into one artery. Therefore, the Willis's circle region-of-interest and the arteriae basilaris region-of-interest are generally determined based on the position of the cerebral tissue, then the potential positions of the four vessels may be estimated based on the above two regions of interest.

The potential positions of the four vessels estimated based on the position of the skull are approximate regions. The centrals of potential end points of the vessels may be obtained by matching these approximate regions with the template of vascular cross-sectional grayscale distribution. The end points of the vessels obtained in this way is more accurate and narrowing the searching range.

Step 204 includes estimating a position of the neck based on the position of the skull, and detecting contrast enhanced images of the neck region layer-by-layer by using an edge detection algorithm and a circular detection operator to obtain potential start points of the vessel in the region of interest.

Step 205 includes positioning and clustering the potential start points and the potential end points by using a clustering algorithm respectively, and selecting positioning points in the maxim cluster as the start points and end points of the vessel.

For four vessels, the start points and end points of the four vessels may be obtained by Step 204 and Step 205. The start points and the end points may be clustered using the clustering algorithm and the mistaken point may be deleted, the points in the maximum cluster are taken as the start point and end point of the vessel. Because there are four vessels in the head and neck region, the respective start point and end point of the left carotid artery, the right carotid artery, the left vertebral artery and the right vertebral artery may be distinguished based on the vascular anatomy and the positional relationship between them.

Step 206 includes calculating a grayscale similarity between each point in the contrast enhanced image and the start points and end points of the vessel, and selecting points with the minimum grayscale similarity to constitute a vascular path.

Because the structure of the vessel in the skull is different from that in the neck, a central of potential start points of the vessel may be determined by Step 204. Step 205 may be further adopted to detect the center of potential start points of the vessel to improve the accuracy of determination.

The grayscale similarity refers to the grayscale difference between each point and the start and end points of the vessel. In practice, the grayscale similarity may be obtained by specifying the minimum grayscale difference between each point and the start and end points and normalizing the specified minimum grayscale difference, then taking its reciprocal to change it into a value between 0 and 1, is taken as the grayscale similarity.

The start point is taken as a reference position. Firstly a point with minimum grayscale difference with the start point in the area adjacent to the start point is selected; and then a point with minimum grayscale difference with the above selected point in the area adjacent to that point is selected. The point with minimum grayscale difference is selected successively until the end point is selected; thereby a vascular path is determined based on all the selected points with minimum grayscale difference.

A path connecting the start point and end point of the vessel is detected by using a priority queue, where the path detected has high similarity with the start and end points of the vessel. For example, firstly, the start point of the vessel is pushed into the priority queue; then each point in the area adjacent to the point to be detected is pushed into the priority queue with iteration, the point has the highest similarity with the start and end points is popped from the priority queue, and the adjacency relation between the point currently popped and the point formerly popped is recorded and the iteration is terminated if the end point is popped. Otherwise, the undetected point in the area adjacent to the popped point is pushed into the priority queue, the iteration continues until the end point of the vessel is popped from the priority queue. According to the order and the adjacency relation between the points popped, the path with the highest similarity from the start point to the end point is obtained by backtracking. That path is the vascular path that connects the start point and the end point of the vessel.

Similarly, the end point may also be taken as the reference position, and the point with the minimum grayscale similarity is determined successively until the start point is determined

Similarly, multiple points of minimum grayscale difference compared with the start point and the end point may also be selected from the areas adjacent to the start point and the end point. A vascular path from the start point to the end point may be determined by using the selected points.

Step 207 includes determining a vascular radius at each point on the vascular path according to grayscale smoothness.

Grayscale smoothness may be used to describe the degree of grayscale difference of all points within the range of vascular radius and may be measured by the grayscale variance within the range of different radius. In this step, the grayscale smoothnesses of a point within the range of different radius are calculated, and then these grayscale smoothnesses are compared with a threshold to select the grayscale smoothnesses larger than the threshold, from which the largest radius is selected as the vascular radius at the point.

Step 208 includes segmenting the contrast enhanced image based on the vascular radius and the vascular path to obtain the vascular region of the vessel.

Step 209 includes combining the subtraction image with the vascular region of the vessel to obtain an angiography image of the vessel.

For four vessels, the contrast enhanced images are combined with the subtraction image on the vascular regions of the four vessels in the head and neck region to cause the subtracted parts of the subtraction image on which the vessels pass through the bone and are maintained.

According to an embodiment of the present disclosure, it is provided a vascular imaging device corresponding to the vascular imaging method according to the first method embodiment of the present disclosure.

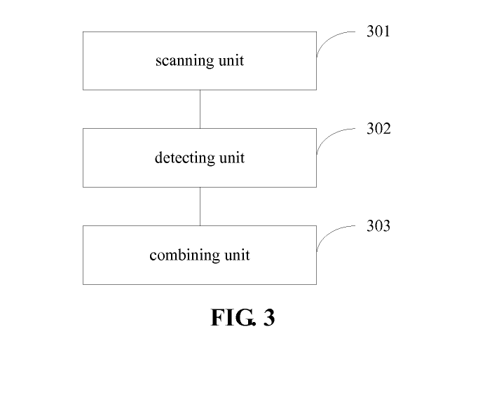

Referring to FIG. 3, a schematic diagram of a vascular imaging device according to an embodiment of the present disclosure is shown. The vascular imaging device includes:

a scanning unit 301, a detecting unit 302 and a fusing unit 303. Hereinafter, each unit will be interpreted and illustrated in combination with the internal structure and the work principle of the device.

The scanning unit 301 is configured to scan a region where a vessel is located to obtain a noncontrast enhanced scan image and a contrast enhanced image and to perform subtraction on the contrast enhanced image based on a bone region in the noncontrast enhanced scan image to obtain a subtraction image.

The detecting unit 302 is configured to detect a vascular region of the vessel in the contrast enhanced image.

The combining unit 303 is configured to combine the subtraction image with the vascular region of the vessel to obtain an angiography image of the vessel.

Optionally, the detecting unit may include:

an estimating sub-unit, configured to estimate a vascular path of the vessel in the contrast enhanced image based on vascular grayscale distribution;

a determining sub-unit, configured to determine a vascular radius at each point on the vascular path according to grayscale smoothness; and

a segmenting sub-unit, configured to segment the contrast enhanced image based on the vascular radius and the vascular path to obtain the vascular region of the vessel.

On the basis of the above detecting unit, optionally, the estimating sub-unit may include:

a first matching module, configured to match the contrast enhanced image and a model of the bone region through which the vessel passes to determine a position of the bone in the contrast enhanced image;

a second matching module, configured to estimate a region of interest where the vessel is located based on the position of the bone, to match contrast enhanced images of the region of interest with a template of vascular cross-sectional grayscale distribution layer-by-layer to determine start points and end points of the vessel; and

a first selecting module, configured to calculate a grayscale similarity between the each point in the contrast enhanced image and the start points and end points of the vessel, and to select a point with minimum grayscale similarity to constitute the vascular path.

On the basis of the above detecting unit, the determining sub-unit may include:

a first calculating module, configured to calculate grayscale smoothnesses within different radiuses with respect to a point on the vascular path; and

a second selecting module, configured to select the largest radius which meets a smoothness threshold as the vascular radius at the point.

On the basis of the above estimating sub-unit, optionally, the second matching module may include:

an establishing sub-module, configured to establish the template of vascular cross-sectional grayscale distribution at least one scale in advance;

a matching sub-module, configured to estimate the region of interest where the vessel is located based on the position of the bone and to obtain a potential position of the vessel in the region of interest by matching the contrast enhanced images of the region of interest with the template of vascular cross-sectional grayscale distribution layer-by-layer; and

a selecting sub-module, configured to position and cluster the potential points of the vessel in the region of interest by using a clustering algorithm and to select points in the maximum cluster as the start points and end points of the vessel based on a positional relationship between the vessel and the bone where the vessel is located.

According to the present disclosure, firstly the region where the vessel is located is scanned to obtain the contrast enhanced image, and subtraction is performed on the contrast enhanced image by using the bone region to obtain the subtraction image; then the vascular region of the vessel in the contrast enhanced image is detected. Thereby the vascular region of the vessel in the contrast enhanced image is determined and the vascular region is the mistakenly subtracted vascular region of the part of the vessel to be detected that passes through or close to the bone; finally, the subtraction image is combined with the vascular region of the vessel to be detected to obtain the angiography image of the vessel.

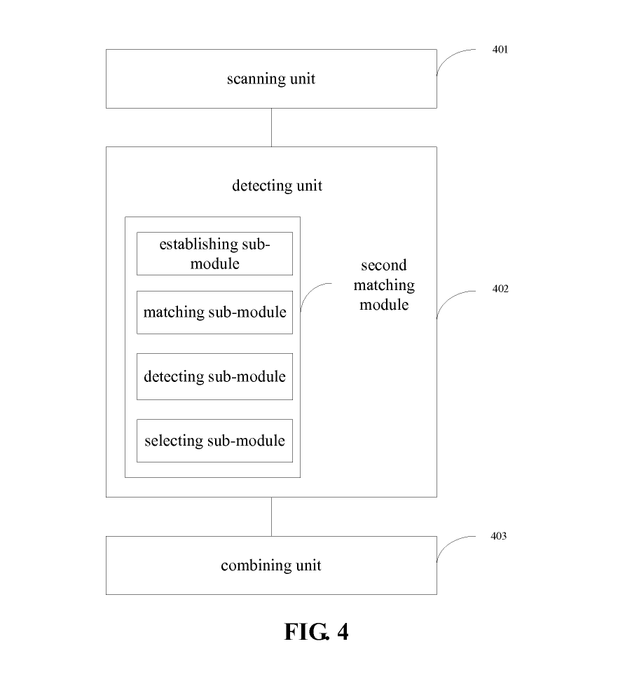

A vascular imaging device is further provided for imaging the vessel in the head and neck region according to the present disclosure, where the second matching module in the establishing sub-module of the above detecting unit differs from that of the first embodiment of the vascular imaging device. Other units and modules are the same as the units and modules in the first embodiment of the vascular imaging device. Referring to FIG. 4, a vascular imaging device according to a second embodiment of the present disclosure is shown. The device includes:

a scanning unit 401, a detecting unit 402 and a fusing unit 403. Except for the second matching module in the detecting unit, other units and modules are the same as the units and modules in the first embodiment. Hereinafter, each unit will be interpreted and illustrated in combination with the internal structure and the work principle of the device.

The detecting unit includes: an estimating sub-unit, a determining sub-unit and a segmenting sub-unit.

The estimating sub-unit includes: a first matching module, a second matching module and a first selecting module. In the case that the vessel is in the head and neck region,

the second matching module includes:

an establishing sub-module, configured to establish a template of vascular cross-sectional grayscale distribution of at least one scale in advance;

a matching sub-module, configured to estimate a region of interest where the vessel is located based on the position of a skull and to match contrast enhanced images of the region of interest with the template vascular cross-sectional grayscale distribution layer-by-layer to determine potential end points of the vessel in the region of interest;

a detecting sub-module, configured to estimate the position of the neck based on the position of the skull and detect contrast enhanced images of the neck region layer-by-layer by using an edge detection algorithm and a circular detection operator to obtain potential start points of the vessel in the region of interest; and

a selecting sub-module, configured to position and cluster the potential start points and the potential end points by using a clustering algorithm and to select the points in a maximum cluster as the start points and end points of the vessel.

According to the embodiment of the present disclosure, the potential start points and potential end points of the vessel are determined respectively through the template of vascular cross-sectional grayscale distribution matching, the edge detection algorithm, the circular detection operator detecting, and the structural characteristic of the bone and the vessel in the neck and head region. In this way, the potential position may be determined more accurately, the calculation complexity may be reduced and the imaging processing rate may be improved.

It should be noted that, in the present disclosure, relational terms such as “first” and “second” are used only to distinguish one entity or operation from the other entity or operation, but not necessarily demand or imply that there is actual relation or order among those entities and operations. Furthermore, the terms “including”, “containing”, or any other variations thereof means a non-exclusive inclusion, so that the process, method, article or device that includes a series of elements includes not only these elements but also other elements that are not explicitly listed, or further includes elements inherent in the process, method, article or device. Moreover, when there is no further limitation, the element defined by the wording “include(s) a . . . ” does not exclude the case that in the process, method, article or device that includes the element there are other same elements.

Moreover, it should also be noted that those skilled in the art could clearly understand that a part or the whole of the process in the embodiments of the above-mentioned method may be implemented by related hardware instructed by computer program. The program may be stored in a computer readable storage medium. When being executed, the program may include the process in the embodiments of the above-mentioned method. The storage medium may be a magnetic disk, an optical disk, a read-only memory (ROM) or a random access memory (RAM) and so on.

The vascular imaging method and apparatus according to the disclosure are described as above. The principles and the embodiments of the invention are described herein by using specific embodiment. However, the description of the above-mentioned embodiments is only for helping to understand the method and the core concept of the invention. Moreover, for the skilled in the art, modifications may be made to the embodiments and the applications in the light of the concept of the invention. As a result, the content in this application should not be understood as limiting the present invention.

1. A vascular imaging method, comprising:
scanning a region where a vessel is located to obtain a noncontrast enhanced scan image and a contrast enhanced image, and performing subtraction on the contrast enhanced image based on a bone region in the noncontrast enhanced scan image to obtain a subtraction image;
detecting a vascular region of the vessel in the contrast enhanced image; and
combining the subtraction image with the vascular region of the vessel to obtain an angiography image of the vessel.

2. The method according to claim 1, wherein detecting a vascular region of the vessel in the contrast enhanced image comprises:
estimating a vascular path of the vessel in the contrast enhanced image based on vascular grayscale distribution;
determining a vascular radius at each point on the vascular path based on grayscale smoothness; and
segmenting the contrast enhanced image based on the vascular radius and the vascular path to obtain the vascular region of the vessel.

3. The method according to claim 2, wherein estimating a vascular path of the vessel in the contrast enhanced image based on vascular grayscale distribution comprises:
matching the contrast enhanced image and a model of the bone region through which the vessel passes to determine a position of a bone in the contrast enhanced image;
estimating a region of interest where the vessel is located based on the position of the bone, matching contrast enhanced images of the region of interest with a template of vascular cross-sectional grayscale distribution layer-by-layer to determine start points and end points of the vessel; and
calculating a grayscale similarity between the each point in the contrast enhanced image and the start points and end points of the vessel, and selecting points with a minimum grayscale similarity to constitute a vascular path.

4. The method according to claim 2, wherein determining a vascular radius at each point on the vascular path according to grayscale smoothness comprises:
calculating the grayscale smoothnesses within different radiuses with respect to the each point on the vascular path; and
selecting the largest radius which meets a smoothness threshold as the vascular radius at the each point.

5. The method according to claim 3, wherein estimating a region of interest where the vessel is located according to the position of the bone, matching contrast enhanced images of the region of interest with a template of vascular cross-sectional grayscale distribution layer-by-layer to determine start points and an end points of the vessel comprises:
establishing the template of vascular cross-sectional grayscale distribution at least one scale in advance;
estimating the region of interest where the vessel is located based on the position of the bone and matching the contrast enhanced images of the region of interest with the vascular cross-sectional grayscale distribution template layer-by-layer to obtain potential positions of the vessel in the region of interest; and
positioning and clustering the potential positions of the vessel in the region of interest by using a clustering algorithm and selecting points in a maximum cluster as the start points and end points of the vessel based on a positional relationship between the vessel to and the bone where the vessel is located.

6. The method according to claim 3, wherein in the case that the vessel is in a head and neck region,
the estimating a region of interest where the vessel is located based on the position of the bone, matching contrast enhanced images of the region of interest with a template of vascular cross-sectional grayscale distribution layer-by-layer to determine start points and an end points of the vessel comprises:
establishing the template of vascular cross-sectional grayscale distribution at least one scale in advance;
estimating the region of interest where the vessel is located based on the position of a skull and matching the contrast enhanced images of the region of interest with the template of vascular cross-sectional grayscale distribution layer-by-layer to determine potential end points of the vessel in the region of interest;
estimating the position of the neck based on the position of the skull and detecting contrast enhanced images of the neck region layer-by-layer by using an edge detection algorithm and a circular detection operator to obtain potential start points of the vessel in the region of interest; and
positioning and clustering the potential start points and the potential end points by using a clustering algorithm and selecting the points in a maximum cluster as the start points and end points of the vessel.

7. The method according to claim 1, further comprising:
displaying the angiography image of the vessel by using three-dimensional volume rendering.

8. A vascular imaging device comprising:
a scanning unit, configured to detect a region where a vessel is located to obtain a noncontrast enhanced scan image and a contrast enhanced image, and to perform subtraction on the contrast enhanced image by using a bone region in the noncontrast enhanced scan image to obtain a subtraction image;
a detecting unit, configured to detect a vascular region of the vessel in the contrast enhanced image; and
a combining unit, configured to combine the subtraction image with the vascular region of the vessel to obtain an angiography image of the vessel.

9. The device according to claim 8, wherein the detecting unit comprises:
an estimating sub-unit, configured to estimate a vascular path of the vessel in the contrast enhanced image based on vascular grayscale distribution;
a determining sub-unit, configured to determine a vascular radius at each point on the vascular path based on grayscale smoothness; and
a segmenting sub-unit, configured to segment the contrast enhanced image based on the vascular radius and the vascular path to obtain the vascular region of the vessel.

10. The device according to claim 9, wherein the estimating sub-unit comprises:
a first matching module, configured to match the contrast enhanced image and a model of the bone region through which the vessel passes to determine a position of a bone in the contrast enhanced image;
a second matching module, configured to estimate a region of interest where the vessel is located based on the position of the bone, and to match contrast enhanced images of the region of interest with a template of vascular cross-sectional grayscale distribution layer-by-layer to determine start points and end points of the vessel; and
a first selecting module, configured to calculate a grayscale similarity between each point in the contrast enhanced image and the start points and end points of the vessel, and to select points with a minimum grayscale similarity to constitute a vascular path.

11. The device according to claim 9, wherein the determining sub-unit comprises:
a first calculating module, configured to calculate the grayscale smoothnesses within different radiuses with respect to the each point on the vascular path; and
a second selecting module, configured to select the largest radius which meets a smoothness threshold as the vascular radius at the each point.

12. The device according to claim 10, wherein the second matching module comprises:
an establishing sub-module, configured to establish the template of vascular cross-sectional grayscale distribution at least one scale in advance;
a matching sub-module, configured to estimate the region of interest where the vessel is located based on the position of the bone and to match the contrast enhanced images of the region of interest with the template of vascular cross-sectional grayscale distribution layer-by-layer to obtain potential positions of the vessel in the region of interest; and
a selecting sub-module, configured to position and cluster the potential position of the vessel in the region of interest by using a clustering algorithm and to select points in a maximum cluster as the start points and end points of the vessel based on a positional relationship between the vessel and the bone where the vessel is located.

13. The device according to claim 10, wherein in the case that the vessel is in a head and neck region,
the second matching module comprises:
an establishing sub-module, configured to establish the template of vascular cross-sectional grayscale distribution at least one scale in advance;
a matching sub-module, configured to estimate the region of interest where the vessel is located based on the position of a skull and to match the contrast enhanced images of the region of interest with the template of vascular cross-sectional grayscale distribution layer-by-layer to determine potential end points of the vessel in the region of interest;
a detecting sub-module, configured to estimate a position of the neck based on the position of the skull and detect contrast enhanced images of the neck region layer-by-layer by using an edge detection algorithm and a circular detection operator to obtain potential start points of the vessel in the region of interest; and
a selecting sub-module, configured to position and cluster the potential start points and the potential end points by using a clustering algorithm and to select points in a maximum cluster as the start points and end points of the vessel.

14. The device according to claim 8, further comprising:
a rendering unit, configured to display the angiography image of the vessel by using three-dimensional volume rendering.
